(19) United States
(12) Patent Application Publication
(10) Pub. No.: US 2008/0214681 A1
(43) Pub. Date: Sep. 4, 2008

(54) TASTE REDUCING COMPOSITIONS AND RELATED METHODS

(75) Inventors: Kenan Sahin, Boston, MA (US); Laura Nattress, Milford, CT (US); David Tisi, Medford, MA (US)

Correspondence Address: OCCHIUTI ROHLICEK & TSAO, LLP, 10 FAWCETT STREET, CAMBRIDGE, MA, 02138, US

(73) Assignee: TIAX, LLC

(21) Appl. No.: 11/841,327

(22) Filed: Aug. 20, 2007

Related U.S. Application Data
(60) Provisional application No. 60/839,052, filed on Aug. 21, 2006.

(51) Int. Cl. A61K 45/00

(52) U.S. Cl. 514/789

(57) ABSTRACT

A method includes contacting a surface in an oral cavity with a first composition capable of reducing a taste of a selected material, and orally administering a second composition separate from the first composition, the second composition including the selected material. The selected material can include a drug, and/or the second composition can further include a drug. In some embodiments, the first and second compositions are included in a kit.

### CLAIM OF PRIORITY

This application claims priority to U.S. Provisional Application Ser. No. 60/839,052 filed on Aug. 21, 2006, and entitled “Bitter Masking Rinse Solution”, which is hereby incorporated by reference.

### TECHNICAL FIELD

The invention relates to taste reducing compositions and related methods. The compositions and methods can be used, for example, to reduce an unwanted flavor of an orally administered composition.

### BACKGROUND

For ease of administration and distribution, many drugs are formed into solid tablets or capsules for oral delivery. Some patients, such as children and the elderly, can experience difficulty in swallowing these solid forms. For these patients, the drugs can be provided in a liquid form, such as solutions, emulsions and suspensions. These liquid forms, however, can expose the patient to an active pharmaceutical ingredient and/or other ingredients in the formulation that taste unpleasant (e.g., bitter). As a result, some patients may not comply with their drug prescriptions.

### SUMMARY

In one aspect, the invention features compositions and related methods that can be used, for example, to reduce an unwanted flavor of an orally administered composition, such as one including a drug. For example, prior to orally taking a drug, the user first washes his oral cavity with a taste reducing solution that is capable of reducing or masking an unwanted taste, such as an unpleasant taste (e.g., a bitter taste, a sour taste, a salty taste, a sweet taste, or a strong taste) of composition containing the drug. Subsequently, at least a portion the taste reducing solution is removed from the oral cavity, and the user orally administers the drug-containing composition, and the unwanted taste is reduced. The compositions and related methods are capable of reducing complaints of poor tasting drugs, which can increase user's compliance with a drug prescription and improve sales of drugs. In addition, the compositions and related methods are easy to administer and use.

In another aspect, the invention features a method, including contacting a surface in an oral cavity with a first composition capable of reducing a taste of a selected material; and orally administering a second composition separate from the first composition, the second composition including the selected material.

Embodiments may include one or more of the following features. The selected material includes a drug. The second composition further includes a drug. The second composition includes a pharmaceutically effective dose of a drug. The second composition includes a liquid. The first composition includes a liquid. The first composition is substantially free of a drug. The method further includes removing at least a portion of the first composition from the oral cavity. Removing the first composition includes swallowing at least a portion of the first composition or expectorating at least a portion of the first composition. The second composition is orally administered less than approximately one minute after removing at least a portion of the first composition from the oral cavity. The second composition is orally administered less than approximately one minute after the surface in the oral cavity is contacted with the first composition. The first composition is capable of reducing the taste of the selected material for at least approximately thirty seconds. The first composition comprises a material capable of forming a barrier between the selected material and taste receptors in the oral cavity. The first composition includes a hydrocolloid. The first composition includes a material selected from the group consisting of an anesthetizing material, a sweetener, and a flavorant.

In another aspect, the invention features a method, comprising contacting a surface of in oral cavity with a first composition capable of reducing a taste of a selected material in a second composition separate from the first composition, the first composition being substantially free of a drug; removing at least a portion of the first composition from the oral cavity; and after removing the first composition from the oral cavity, orally administering the second composition, the second composition including a drug. The first and second compositions can include a liquid.

In another aspect, the invention features a kit, including a first composition capable of reducing a taste of a selected material; and a second composition separate from the first composition, the second composition including the selected material.

Embodiments may include one or more of the following features. The selected material includes a drug. The second composition further includes a drug. The second composition includes a pharmaceutically effective dose of a drug. The second composition includes a liquid. The first composition includes a liquid. The first composition is substantially free of a drug. The first composition is capable of reducing the taste of the selected material for at least approximately thirty seconds. The first composition includes a material capable of forming a barrier between the selected material and taste receptors in an oral cavity. The first composition includes a hydrocolloid. The first composition includes a material selected from the group consisting of an anesthetizing material, a sweetener, and a flavorant.

In another aspect, the invention features a kit including a first composition capable of reducing a taste of a selected material, the first composition being substantially free of a drug; and a second composition separate from the first composition, the second composition including the selected material. The second composition can include a drug. The first and second compositions can include a liquid.

Other aspects, features and advantages will be apparent from the description of the embodiments thereof and from the claims.

### DETAILED DESCRIPTION

A method of orally administering a drug to a user includes contacting a surface in the user's oral cavity with a first composition capable of reducing an unwanted taste of a second composition containing the drug, and then separately orally administering the second composition. For example, the user first washes his oral cavity with a taste reducing solution that is capable of reducing or masking an unwanted taste, such as a bitter taste, of a drug-containing composition. The oral cavity is washed for a time sufficient to reduce or to mask the unwanted taste, and subsequently, at least a portion (e.g., all of) the taste reducing solution is removed (e.g., by swallowing or expectorating the taste reducing solution). Then, the user orally administers the drug-containing composition (e.g., a solution), and the unwanted taste is reduced.

The taste reducing composition is capable of reducing one or more unpleasant tastes from the drug-containing composition. The unpleasant taste(s) can come from the drug itself and/or one or more other ingredients in the drug-containing composition. The taste reducing composition is typically substantially free of or has less than a pharmaceutically effective dose of one or more drugs contained in the drug-containing composition. In some embodiments, the taste reducing ability of the taste reducing composition lasts for a short period (e.g., less than approximately ten minutes, or less than approximately five minutes), so as to not affect the user's taste perception longer than needed for the administration of the drug-containing composition. In some embodiments, the taste reducing composition is wholly in liquid form. In other embodiments, the taste reducing composition includes a liquid and another phase (e.g., a suspension).

During administration, a user can take an effective portion (e.g., approximately one ounce (28 ml)) of the taste reducing composition and place it in his mouth. The composition is then agitated (e.g., for approximately five to approximately thirty second), such that it contacts a surface (e.g., the entire surface including the tongue) of the oral cavity. Subsequently, at least a portion of the taste reducing composition can be expectorated and/or swallowed, at the user's preference.

In some embodiments, the taste reducing composition includes an aqueous solution including a hydrocolloid, a mild anesthetizing agent, a sweetener, and a flavorant.

The hydrocolloid is capable of acting as a physical barrier between taste receptors in the oral cavity and the compounds that can cause an unwanted (e.g., bitter) taste. The hydrocolloid is capable of preventing the perception of unwanted taste by forming a temporary barrier (e.g., a film) that reduces the transmission and contact of unwanted tasting compounds to the taste receptors. Examples of hydrocolloids include those that can produce an effective, fast acting, and short-lived effect in the mouth, such as agar alginate, gum arabic, carrageenan, casein, cellulose derivatives, conjac, furcelleran, galactomannan, gelatin, gellan, guar gum, locust bean gum, maltodextrin, pectin and derivatives, polyvinylalcohol (PVA), polyvinylpyrrolidone (PVP), starch and derivatives, tragacanth, or xanthan gum. PVP, also referred to as povidone, is available under the tradenames Plasdone®, Povidone®, Polyplasdone®, and Kollidon for a cross-linked PVP, from ISP Technologies (Wayne, N.J.) and BASF (Florham Park, N.J.). The concentration of the hydrocolloid can depend on their barrier forming (e.g., bitter masking) ability. In some embodiments, the concentration of the hydrocolloid ranges from approximately 0.1% by weight to approximately 25% by weight The concentration of the hydrocolloid can be greater than or equal to, by weight, approximately 0.1%, approximately 5%, approximately 10%, approximately 15%, or approximately 20%; and/or less than or equal to, by weight, approximately 25%, approximately 20%, approximately 15%, approximately 10%, or approximately 5%. Conjac, for example, can be effective at concentrations of approximately 0.1% by weight to approximately 2% by weight A more soluble, less viscous hydrocolloid, such as gum arabic, can be effective at higher concentrations, e.g., approximately 20% by weight to approximately 25% by weight. The taste reducing composition can include one hydrocolloid or multiple different hydrocolloids.

The mild anesthetizing agent can serve a dual purpose of inhibiting the unwanted (e.g., bitter) taste mechanism and creating a trigeminal stimulation to divert the user from the unwanted taste. Some anesthetizing agents can also contribute to the ultimate flavor of the taste reducing solution. Examples of anesthetizing agents include benzocaine, camphor, dyclonine hydrochloride, menthol, phenol, eucalyptol, peppermint oil, methyl salicylate, bornyl acetate, capsaicin, phenol, hexylresorcinol, salicyl alcohol, benzyl alcohol, dyclonine, dibucaine, buticaine, cetylpyridinium chloride, diperidon, clove oil, bupivacaine, butambenpicrate, chlorprocaine, cocaine, dibucaine, dimethisoquin, dyclonine, etidocaine, hexylcaine, hexylresorcinol, ketarine, lidocaine, mepivacaine, phenol, phenolate, pramoxine, procaine, ropavacaine, tetracaine, tripelennamine, xylocaine, and pharmaceutically acceptable salts thereof (e.g., dimethisoquin hydrochloride, pramoxine hydrochloride). In some embodiments, the concentration of the anesthetizing agent can range from approximately 0.05% by weight to approximately 3.0% by weight, depending, for example, on the agent's taste masking efficacy. The concentration of the anesthetizing agent can be greater than or equal to, by weight, approximately 0.05%, approximately 0.5%, approximately 1.0%, approximately 1.5%, approximately 2.0%, or approximately 2.5%, and/or less than or equal to, by weight, approximately 3.0%, approximately 2.5%, approximately 2.0%, approximately 1.5%, approximately 1.0%, or approximately 0.5%. For example, a concentration of menthol from approximately 125 to approximately 400 ppm can effectively mask or reduce an unwanted taste. The taste reducing composition can include one anesthetizing agent or multiple different anesthetizing agents.

One or more sweeteners and/or flavorants can be used to suppress unwanted flavors and/or to enhance the masking or taste reducing functionality of other compounds in the taste reducing composition. The sweetener(s) and/or flavorant(s) can be caloric and/or non-caloric, and can include those approved for use in pharmaceutical formulations. Examples of sweeteners include, sucrose, fructose, dextrose, xylitol, mannitol, aspartame, sucralose, saccharin sodium, and acesulfame potassium, and examples of flavorants include, orange, pineapple, grape, strawberry, cherry, peach, raspberry, peppermint, spearmint, fruit punch, and bubblegum.

In some embodiments, the taste reducing composition further includes other compounds that are used in pharmaceutical products and/or nutritional foods. Examples of these compounds include preservatives, colors, processing aids, and ingredients that enhance the solubility of any compound in the composition.

The drug-containing composition includes a pharmaceutically effective dose of one or more drugs. The drug-containing composition can include solely of one or more drugs, or the composition can include one or more drugs combined with other ingredients (such as a carrier, a solubilizing agent, an excipient, a preservative, a flavorant, and other taste enhancers). In some embodiments, the drug-containing composition is wholly in liquid form. In other embodiments, the drug-containing composition includes a liquid and another phase (e.g., a suspension). Examples of unwanted and/or unpleasant tasting drugs include antibiotics, HIV/AIDS drugs, cough medicine, cold medicine, antihistamines, drugs that treat asthma, anticancer drugs, antacids, and analgesics. In some embodiments, the drug-containing composition is free of a material capable of forming a film, e.g., a film that can mask the taste of the drug(s). During administration, after removing at least a portion of the taste reducing composition from the oral cavity, the drug-containing composition can be taken orally immediately or within the time period that the taste reducing composition remains effective (e.g., within approximately five minutes, within approximately three minutes, or within approximately one minute).

The taste reducing composition and the drug-containing composition can be packaged in a kit. The kit can include, for example, instructions to take the taste reducing composition first, to remove the taste reducing composition, and then to take the drug-containing composition within a specified time period. In some embodiments, the taste reducing composition and the drug-containing composition can be stored in separate chambers of a device (e.g., a dual-chambered syringe) that allows the compositions to be orally delivered separately and in a selected sequence.

While a number of embodiments have been described, the invention is not so limited.

For example, in some embodiments, the drug-containing composition is administered in non-liquid form, such a chewable tablet.

The first composition and/or the second composition can be provided and/or administered in non-liquid form (e.g., as a solid and/or a powder). In some embodiments, the composition(s) can be mixed in (e.g., dissolved in) a liquid (such as water or another beverage) prior to administering the composition(s) to a user.

Still other embodiments are within the scope of the following claims.

What is claimed is:

1. A method, comprising:
contacting a surface in an oral cavity with a first composition capable of reducing a taste of a selected material; and
orally administering a second composition separate from the first composition, the second composition comprising the selected material.

2. The method of claim 1, wherein the selected material comprises a drug.

3. The method of claim 1, wherein the second composition further comprises a drug.

4. The method of claim 1, wherein the second composition comprises a pharmaceutically effective dose of a drug.

5. The method of claim 1, wherein the second composition comprises a liquid.

6. The method of claim 1, wherein the first composition comprises a liquid.

7. The method of claim 1, wherein the first composition is substantially free of a drug.

8. The method of claim 1, further comprising removing at least a portion of the first composition from the oral cavity.

9. The method of claim 8, wherein removing the first composition comprises swallowing at least a portion of the first composition or expectorating at least a portion of the first composition.

10. The method of claim 8, wherein the second composition is orally administered less than approximately one minute after removing at least a portion of the first composition from the oral cavity.

11. The method of claim 1, wherein the second composition is orally administered less than approximately one minute after the surface in the oral cavity is contacted with the first composition.

12. The method of claim 1, wherein the first composition is capable of reducing the taste of the selected material for at least approximately thirty seconds.

13. The method of claim 1, wherein the first composition comprises a material capable of forming a barrier between the selected material and taste receptors in the oral cavity.

14. The method of claim 1, wherein the first composition comprises a hydrocolloid.

15. The method of claim 1, wherein the first composition comprises a material selected from the group consisting of an anesthetizing material, a sweetener, and a flavorant.

16. A method, comprising:
contacting a surface of in oral cavity with a first composition capable of reducing a taste of a selected material in a second composition separate from the first composition, the first composition being substantially free of a drug;
removing at least a portion of the first composition from the oral cavity; and
after removing the first composition from the oral cavity, orally administering the second composition, the second composition comprising a drug.

17. The method of claim 16, wherein the first and second compositions comprise a liquid.

18. A kit, comprising:
a first composition capable of reducing a taste of a selected material; and
a second composition separate from the first composition, the second composition comprising the selected material.

19. The kit of claim 18, wherein the selected material comprises a drug.

20. The kit of claim 18, wherein the second composition further comprises a drug.

21. The kit of claim 18, wherein the second composition comprises a pharmaceutically effective dose of a drug.

22. The kit of claim 18, wherein the second composition comprises a liquid.

23. The kit of claim 18, wherein the first composition comprises a liquid.

24. The kit of claim 18, wherein the first composition is substantially free of a drug.

25. The kit of claim 18, wherein the first composition is capable of reducing the taste of the selected material for at least approximately thirty seconds.

26. The kit of claim 18, wherein the first composition comprises a material capable of forming a barrier between the selected material and taste receptors in an oral cavity.

27. The kit of claim 18, wherein the first composition comprises a hydrocolloid.

28. The kit of claim 18, wherein the first composition comprises a material selected from the group consisting of an anesthetizing material, a sweetener, and a flavorant.

29. A kit, comprising:
a first composition capable of reducing a taste of a selected material, the first composition being substantially free of a drug; and
a second composition separate from the first composition, the second composition comprising the selected material.

30. The kit of claim 29, wherein the second composition comprises a drug.

31. The kit of claim 29, wherein the first and second compositions comprise a liquid.
